(19) United States
(12) Patent Application Publication
(10) Pub. No.: US 2012/0123459 A1
(43) Pub. Date: May 17, 2012

(54) SAFETY LANCETS HAVING ANGLED GRIPS

(75) Inventor: Christopher J. RUF, Marietta, GA (US)

(73) Assignee: FACET TECHNOLOGIES, LLC, Kennesaw, GA (US)

(21) Appl. No.: 13/293,610

(22) Filed: Nov. 10, 2011

Related U.S. Application Data
(60) Provisional application No. 61/412,574, filed on Nov. 11, 2010.

(51) Int. Cl. A61B 17/32

(52) U.S. Cl. 606/182

(57) ABSTRACT

A single-use safety lancet having a housing, a triggering mechanism, and at least one grip. The housing contains a drive system and includes a proximal end and a distal end. The triggering mechanism is coupled to the proximal end of the housing and is operable to activate a lancet stroke. The at least one grip extends from the housing and is operable to permit user interaction therewith.

### CROSS-REFERENCE TO RELATED APPLICATION

This application claims the benefit of U.S. Provisional Patent Application Ser. No. 61/412,574, filed Nov. 11, 2010, the entirety of which is hereby incorporated herein by reference for all purposes.

### TECHNICAL FIELD

The present invention relates generally to the field of lancing devices, and more particularly to single-use lancing devices for obtaining a sample of blood for a variety of tests.

### BACKGROUND OF THE INVENTION

Samples of blood are sometimes collected from a human or animal subject. For example, many diabetics periodically monitor their blood glucose level by collecting a small blood sample from their fingertip, forearm, or other body part. In addition, small blood samples are commonly collected prior to accepting blood from a donor at blood drives or blood banks in order to determine the donor's blood type and/or to screen potential donors for anemia, diseases, or other conditions.

Lancing devices are typically used to pierce the skin of a subject in order to obtain a sampling of blood, interstitial fluid, and/or other bodily fluids. Typically, a lancing device incorporates a spring-driven lancet and some type of triggering mechanism to release stored energy from the spring to drive the sharp edge/point of the lancet to penetrate the subject's skin. In particular, single-use disposable lancing devices are typically employed to reduce the risks of disease transmission through blood transfer and/or other bodily fluids. These single use devices reduce these risks by preventing the reuse of needles, lancets, or other sharp objects that become contaminated with blood or other bodily fluids, being intended for disposal after use with an individual test subject.

However, in an effort to keep material use and cost down, single-use safety lancets are typically small and simple, and as a result can be difficult for a user to grip and control. These devices also typically have gripping surfaces that are small and/or slippery. Yet, since a user must maintain good control of these small lancet devices in order to manipulate a firing mechanism and/or press the device into a subject's skin to activate, a firm grip is important to ensuring proper and safe operation of the device. Commercially available single-use lancing devices typically are fabricated of molded resin, which can be slick and provide a poor gripping surface. Some known devices, for example as depicted in FIG. 1, include ribs for gripping, but they can be uncomfortable and/or create painful pressure points for some users, while not significantly improving some users' ability to grip the device.

Accordingly, it can be seen that needs exist for single-use lancing devices that are easier to grip, aim, and activate by a user. It is to the provision of improved single-use lancing devices meeting these and other needs that the present invention is primarily directed.

### SUMMARY OF THE INVENTION

In example embodiments, the present invention provides for safety lancets that include one or more grips that extend from the body of the lancet device to aid a user in handling and operating the same. The one or more grips included with the safety lancets of the present invention add significant gripping capabilities above and beyond that known in the prior art. Indeed, the present invention requires minimal user effort to obtain and maintain a confident and comfortable grip when operating the same to obtain a blood (and/or other fluid) sample from a subject.

For example, in one aspect, the present invention relates to a single-use safety lancet having a housing, a triggering mechanism, and at least one grip. The housing contains a drive system and includes a proximal end and a distal end. The triggering mechanism is coupled to the proximal end of the housing and is operable to activate a lancet stroke. The at least one grip extends from the housing and is operable to permit user interaction therewith.

In another aspect, the invention relates to a pressure activated single-use safety lancet for piercing a subject's skin. The safety lancet includes an elongated body and at least one grip. The body generally houses a spring-driven lancet apparatus. The at least one grip extends from the elongated body and is operable to engage a user's finger and provides a surface for applying pressure in a direction parallel to the elongation of the body. Optionally, the safety lancet includes a pair of opposed grips.

In another aspect, the invention relates to a method for piercing the skin of a subject using a single-use safety lancet that has an elongated body housing a spring-driven lancing apparatus and at least one grip extending from the elongated body. The method includes positioning the safety lancet with respect to a preferred location on the skin of the subject and gripping the at least one grip with at least one human finger. The method also includes activating the spring-driven lancing apparatus to be driven toward the skin of the subject.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a perspective view of a known lancing device.

FIGS. 2A-2F depict various views of a safety lancet according to an example embodiment of the present invention.

FIGS. 3A-3G depict various views of the safety lancet of FIGS. 2A-2F, shown without its protective endcap.

FIGS. 4A-4G depict various views of a safety lancet according to another example embodiment of the present invention.

FIGS. 5A-5H depict various views of the safety lancet of FIGS. 4A-4G, shown without its protective endcap.

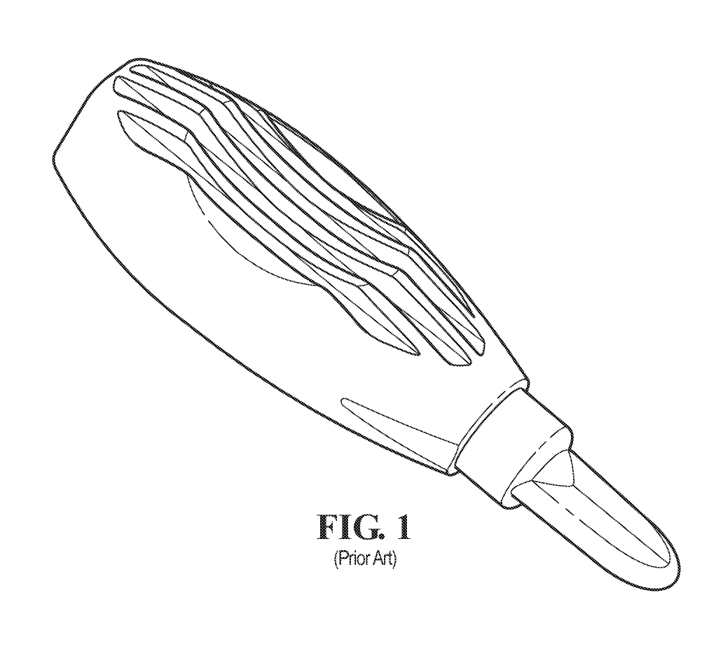

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

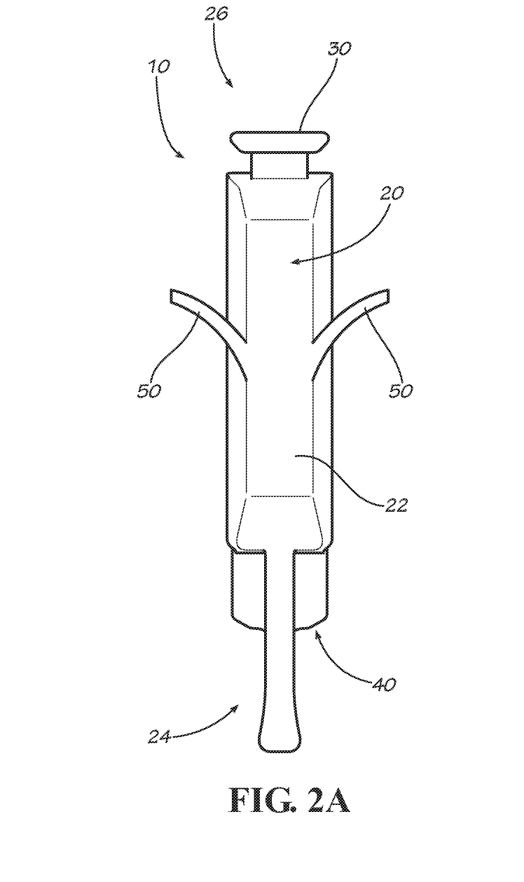

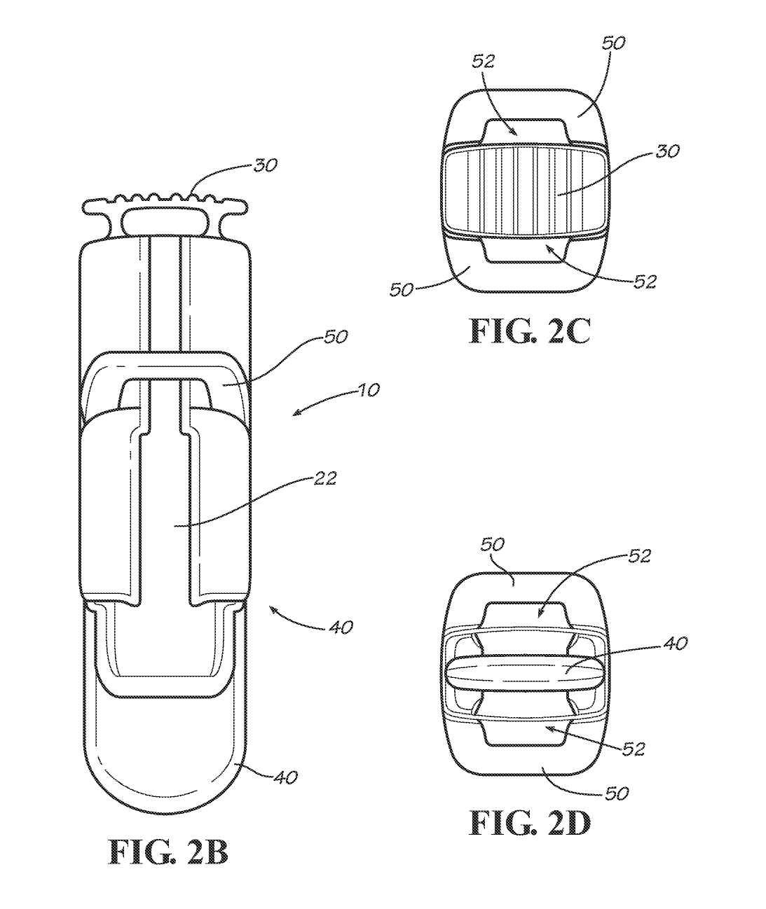

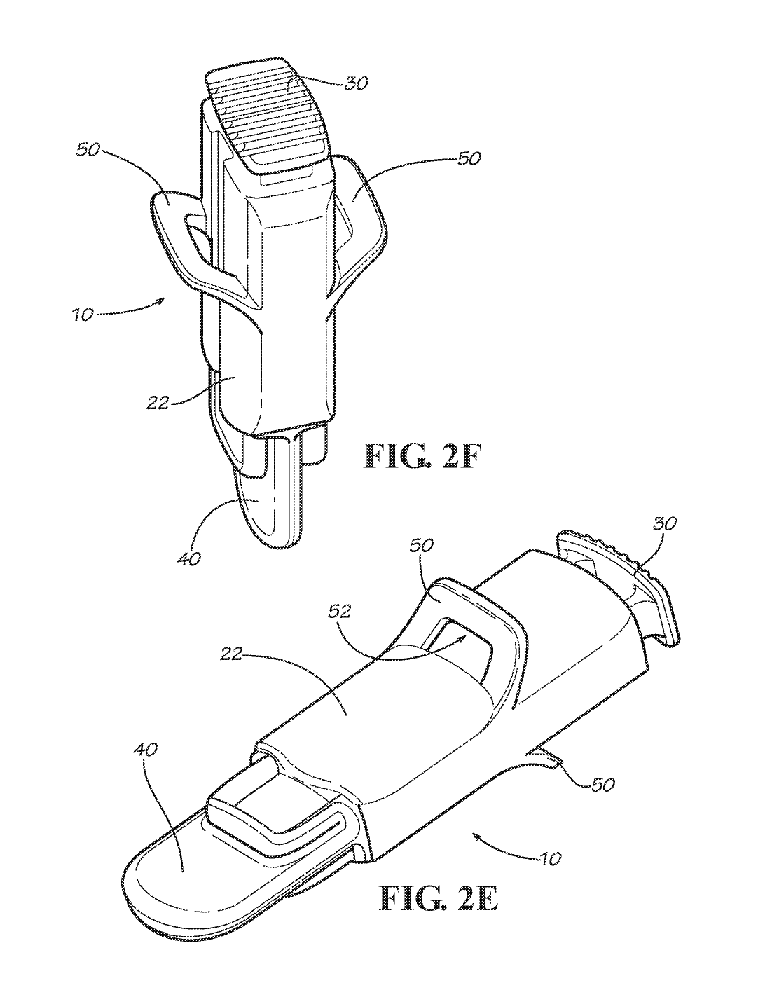

With reference now to the drawing figures, wherein like reference numbers represent corresponding parts throughout the several views, FIGS. 2A-5H depict various embodiments of a safety or single-use lancet according to example embodiments of the present invention. Generally, the safety lancets of the present invention include one or more grips or contact wings that extend from the body of the lancet device to aid a user in handling and operating the same. The one or more grips included with the safety lancets of the present invention are believed to add significantly improved gripping and control capabilities as compared to previously known devices. Indeed, the present invention requires minimal user effort to obtain and maintain a confident and comfortable grip when operating the same to obtain a blood (and/or other fluid) sample from a subject.

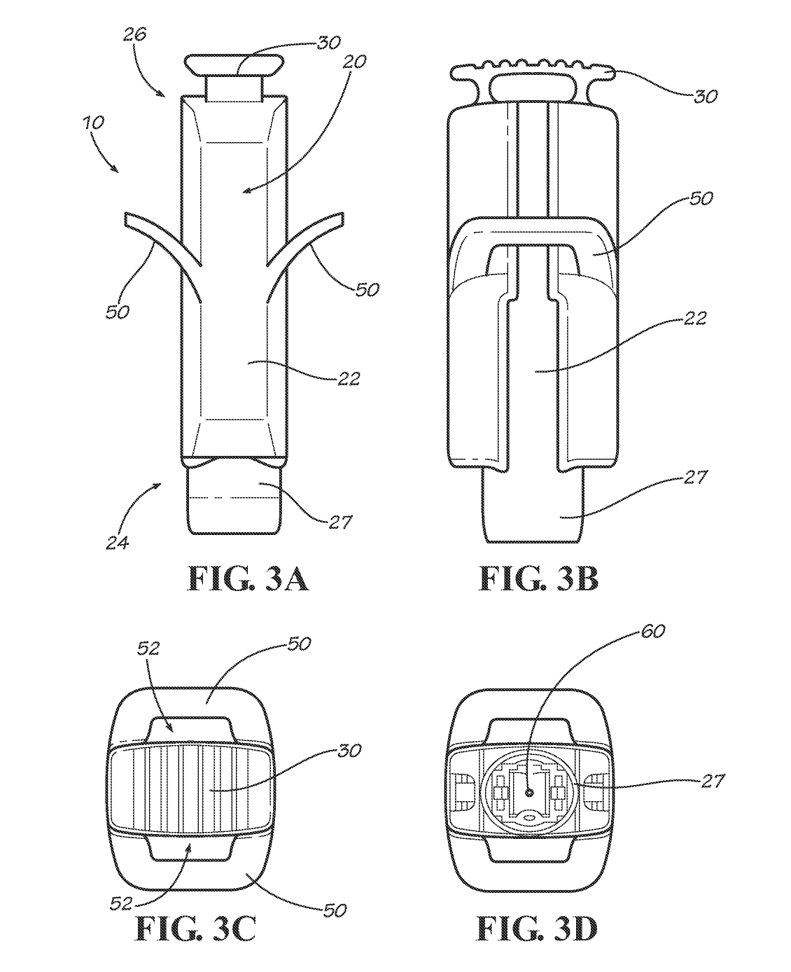

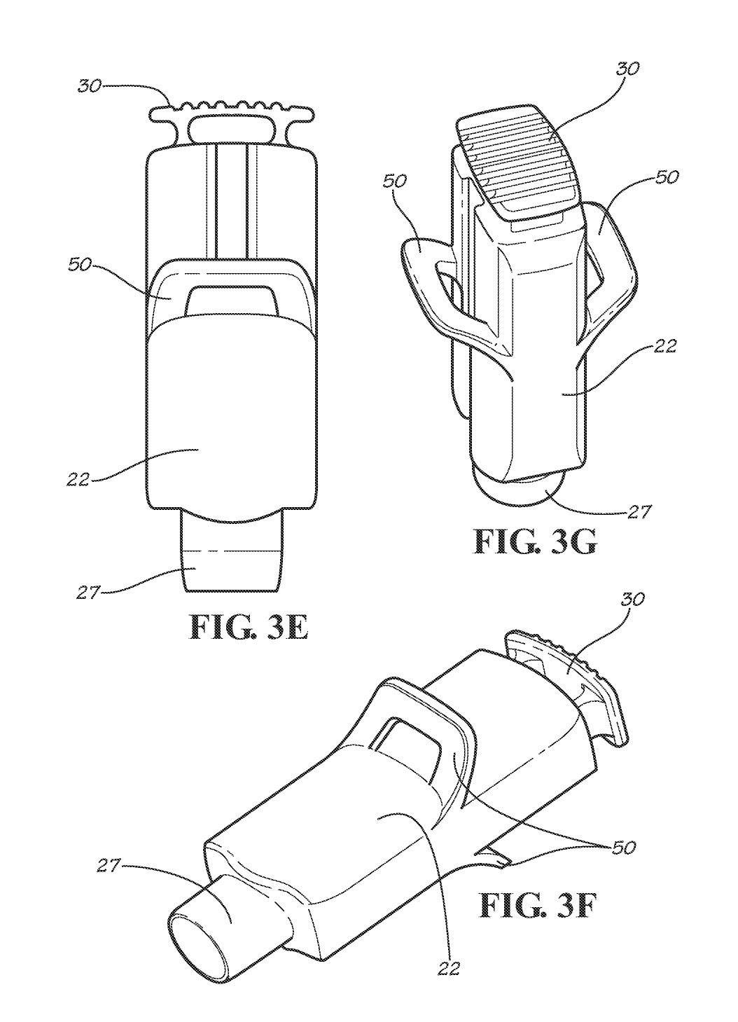

With particular reference now to FIGS. 2A-2F, a safety lancet 10 according to a first example embodiment of the present invention is generally shown therein. The safety lancet 10 shown in FIGS. 2A-2F is of the type of lancing device that is spring-driven and trigger-activated. In use, a user releases a trigger mechanism causing energy stored in the drive spring to drive a sharp edge or point of a lancet along a lancing stroke in order to penetrate a subject's skin. As shown in the drawing figures, the safety lancet 10 generally includes a housing 20 and a trigger mechanism 30. The safety lancet 10 can also include a protective endcap or shield 40 to cover the lancet opening of the housing and prevent inadvertent contact with the sharp edge or point of the lancet and/or to maintain sterility. FIGS. 3A-3G depict various views of the safety lancet 10 shown in FIGS. 2A-2F with the cap 40 removed. In example embodiments, at least one grip or contact wing 50 extends from the housing 10, however in alternative embodiments, two or more grips or contact wings 50 extend from the housing. In preferred example embodiments, two symmetric but mirror-image opposed grips 50 extend from the housing in transversly opposite directions directly across from one another as depicted in the drawing figures.

The housing 20 generally defines an elongated body 22 having a lancet structure and drive mechanism disposed therein. The body 22 includes a distal or forward end 24 with a lancet opening therein for engaging the skin of a subject, and a rearward or proximal end 26. The trigger mechanism 30 used to initiate a lancet stroke (and release the energy stored within an internal spring) is shown disposed at the proximal end 26 of the body 22. However, in alternative embodiments, the trigger mechanism 30 can be employed in various locations along the housing 20 as desired by a user. For example, in other embodiments, the triggering mechanism can be disposed near the distal end 24 or any other comfortable and/or convenient location along the housing body. The depicted triggering mechanism 30 is a push-type button, however, other embodiments can utilize triggers or other conventional initiating mechanisms as are well known in the art. The lancet cap 40, when utilized, is generally positioned at the distal end 24 as shown in FIGS. 2A-2I. Beneath the lancet cap 40, and protected within a collar 27 fixedly extending from the body 22, the safety lancet includes at least one lancet 60 as best seen in FIG. 3D. In operation, the lancet cap 40 is removed prior to use of the safety lancet 10 for those embodiments configured with such. The cap 40 can be secured to the housing 20 through a variety of methods, for example friction fit, corresponding threaded surfaces, snap fit, molding or other methods well known in the art. The cap 40 can be removed from the housing 20 by a variety of methods, for example pulling, unscrewing, twisting or other methods well known in the art. It shall be noted that the present invention includes embodiments of safety lancets that do not include such a lancet cap, and/or that include a foil or film cover or other safety/sterility barriers.

The device of the present invention includes at least one unique grip 50 that aids a user in handling and using the safety lancet 10 as described herein. As shown in FIGS. 2A-3G, example embodiments include a pair of opposed grips or contact wings 50 that extend from the housing 20 along the body 22 between the distal end 24 and the proximal end 26. As best seen in FIG. 2A, the grips 50 extend from the housing 20 at an oblique angle and reach towards the triggering mechanism 30. In addition, in example embodiments of the present invention the grips 50 comprise a curved or arcuately contoured flange to more comfortably and positively be engaged by a user's finger(s) and further provide for increased gripping capabilities. Alternate embodiments can utilize substantially straight or planar gripping surfaces arranged at an oblique angle relative to the adjacent sides of the housing. In alternate embodiments, the grips extend substantially perpendicular from the housing to form a generally “T” or cross shaped body. In such alternative embodiments, the substantially perpendicular grips can extend from the housing at some point between the distal end 24 and the proximal end 26 (e.g., at a midpoint between the distal end and proximal end, or otherwise disposed between the same). In still other example embodiments, at first grip 50 can extend in a substantially perpendicular nature from the housing, while a second grip can extend obliquely from the same. The grips can be directly opposite one another, or alternatively can be offset from one another along the length of the body. Each of the grips or wings preferably comprises a single flange or panel attached to or integrally formed with the housing along one edge thereof, and extending outwardly from the housing with an outer free edge directed away from the housing and proximally and distally directed surfaces oriented generally transversely or obliquely relative to the adjacent surface of the housing. The grips 50 can be of solid construction or can include one or more apertures 52, as shown in FIGS. 2E and 2F, to reduce material requirements and costs, and/or for improved gripping characteristics. Alternatively or additionally, to further aid in a user's ability to grip the safety lancet 10 and handle the same in use, the grips 50 can comprise raised or recessed ribs, slits, knurls, dimples, adhesive(s), or other surface treatment to provide a more complete gripping surface.

In operation, the safety lancet 10 of the present invention provides for a firm contact grip and superior control of the device without creating uncomfortable pressure points that can result from some known devices. To utilize the safety lancet 10, a user places one or more fingers onto or around the grips or contact wings 50 and places his/her thumb or any remaining finger on the triggering mechanism 30 to handle and manipulate the safety lancet. Once the user has oriented the safety lancet 10 in a desired position over the skin of a subject, the user can utilize the grips 50 to provide stable leverage, against which the user can press and activate the triggering mechanism 30.

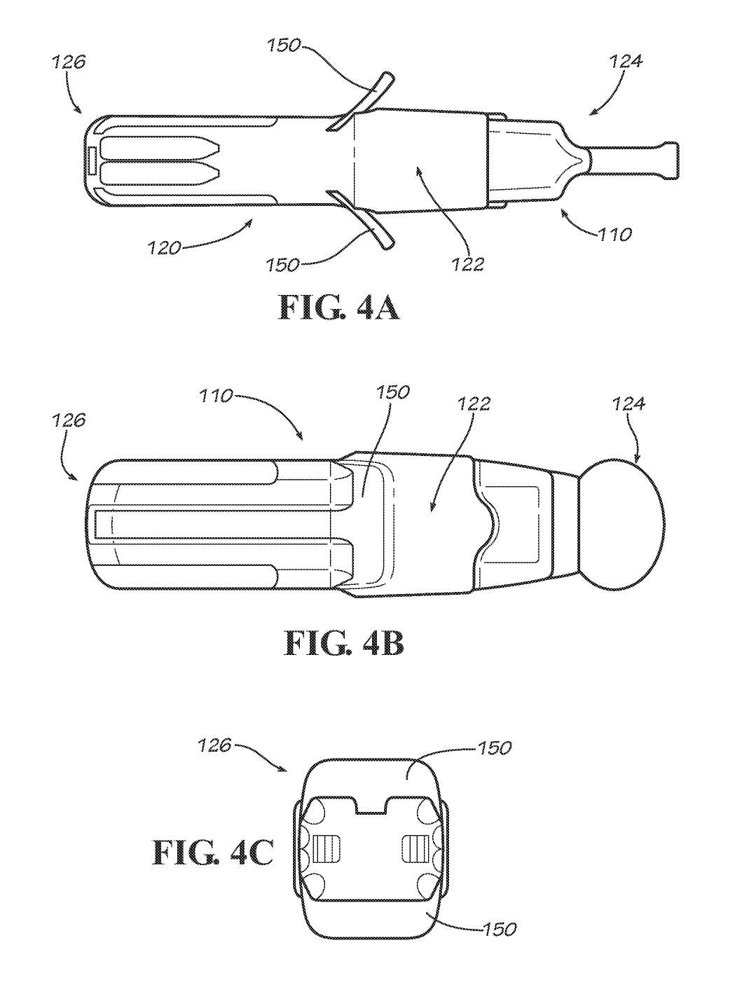

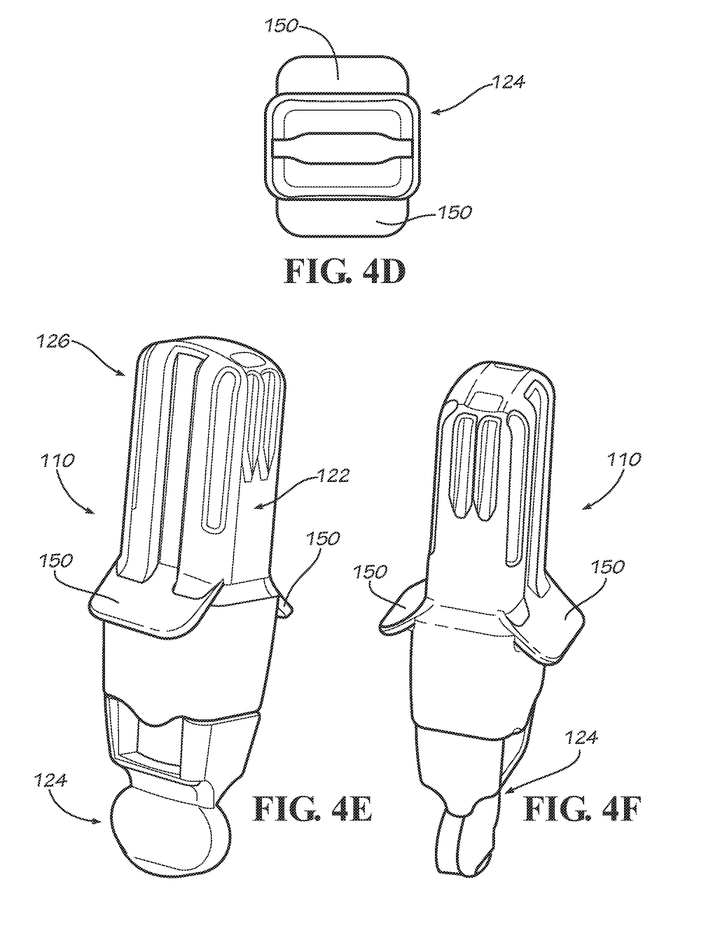

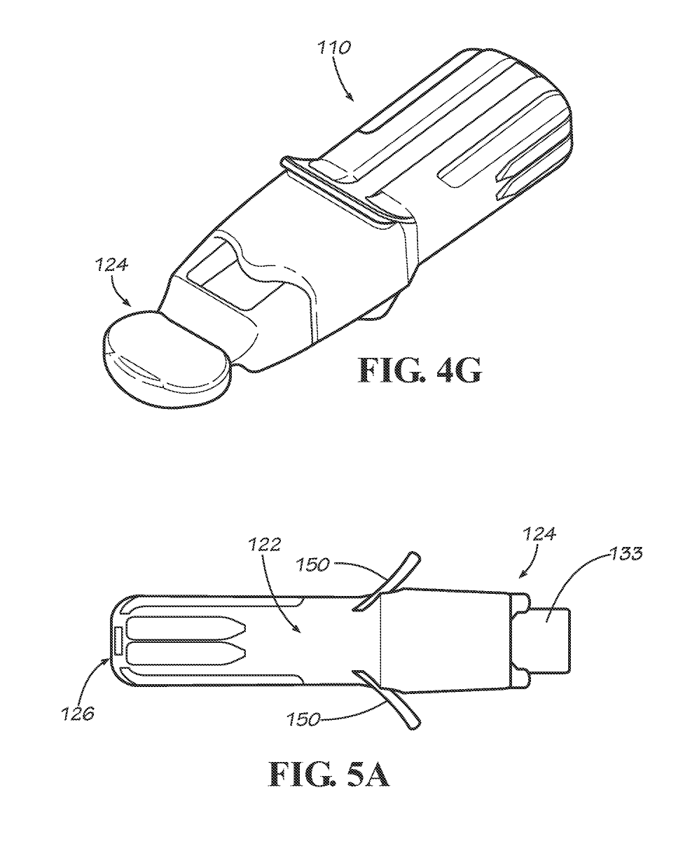

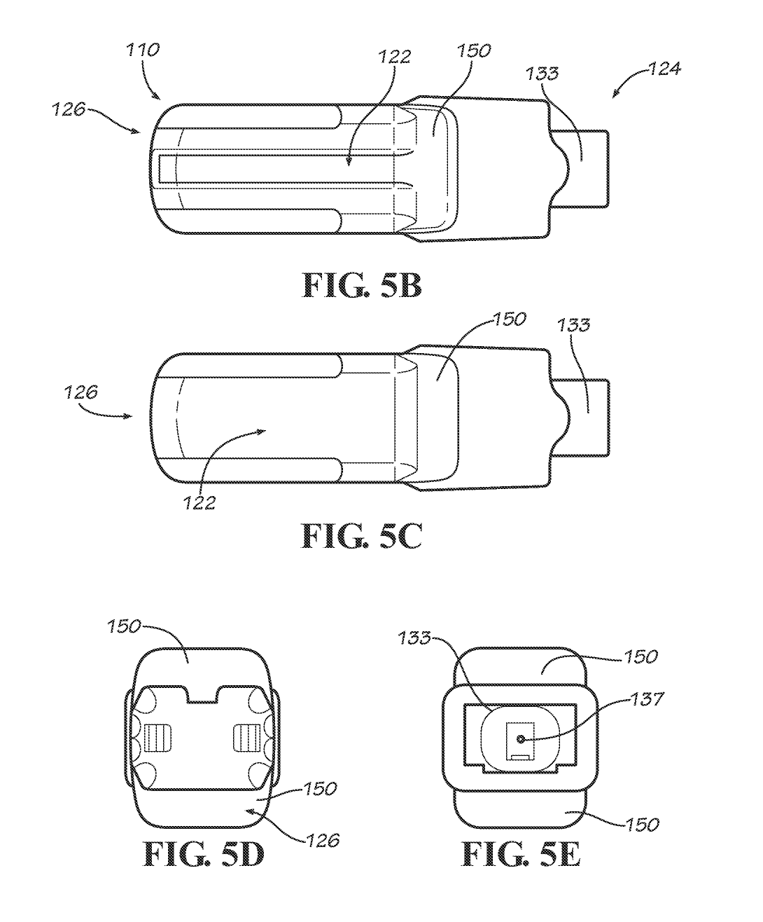

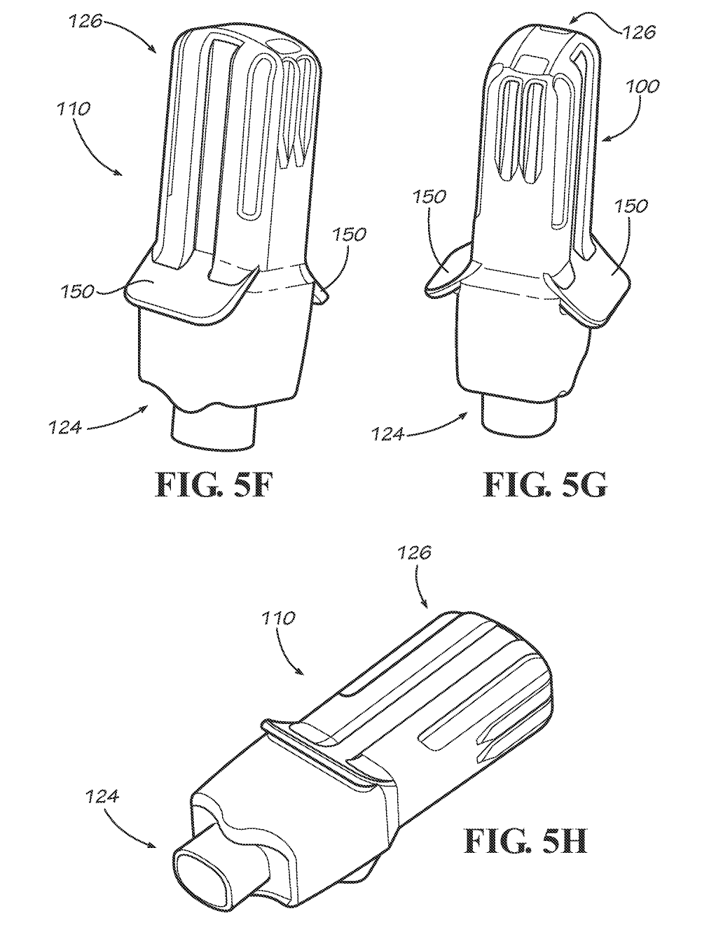

With reference now to FIGS. 4A-5H, another example embodiment of a safety lancet 110 according to the present invention is shown and described. Safety lancet 110 is of the type of lancing device that is spring-driven and pressure-activated by a user to release energy stored in the spring to drive a sharp edge or point of a lancet into a subject's skin to penetrate the same. Example forms of this safety lancet 110 include a pair of opposed grips or contact wings 150 that extend from an elongated body 122 of the housing 120 in an orientation towards the distal end 124. Otherwise, grips 150 are similar in both their construction and function to the previously-described embodiments. Because safety lancet 110 is pressure activated (as opposed to trigger activated), a user operating the same generally uses one or more fingers to push on the grips 150 and/or utilize the grips to stabilize the lancet when orienting it over the skin of a targeted subject. To activate the lancet (and trigger the initiation of the spring-driven lancing stroke) after orientation of the device, the user can apply pressure from his/her thumb or palm of hand to the proximal end 126 of the safety lancet and/or apply pressure to the grips 150 to press the lancet against the skin of the test subject. Multiple variations to the safety lancet 110 and/or grips 150 can be employed as discussed above in regards to lancet 10 and grips 50. Beneath the lancet cap at the distal end 124, and protected within a collar 133 retractably extending from the body 122, the safety lancet includes a lancet 137 as best seen in FIG. 5E. In operation, when a user aligns the lancet with a location on the skin, the user applies a downward pressure onto the grips 150. The pressure on the collar 133 retracts the collar to expose the sharp tip 137 for lancing and/or triggers release of the spring-driven drive mechanism of the device to propel the lancet along a lancing stroke and thus carry out the lancing procedure.

The safety lancets of the various embodiments of the present invention can be fabricated from a variety of materials, including, but not limited to, plastics, resins, rubbers, other polymers, metals, ceramics, or a combination of such. In addition, the grips 50, 150 can be coated with one or more surface enhancers to improve the gripping qualities of the same.

While the invention has been described with reference to preferred and example embodiments, it will be understood by those skilled in the art that a variety of modifications, additions and deletions are within the scope of the invention, as defined by the following claims.

What is claimed is:

1. A single-use safety lancet comprising:
a housing substantially enclosing a lancet and a drive system for driving the lancet therein, the housing having a proximal end and distal end;
a triggering mechanism coupled to the housing for activating the drive system to drive the lancet along a lancet stroke; and
at least one contact wing extending from the housing forming a pressure surface for assisting in gripping the housing.

2. The single-use safety lancet of claim 1, wherein the at least one contact wing comprises a pair of opposed contact wings.

3. The single-use safety lancet of claim 1, wherein the at least one contact wing is curved.

4. The single-use safety lancet of claim 1, wherein the at least one contact wing extends from the housing at an oblique angle.

5. The single-use safety lancet of claim 4, wherein the at least one contact wing extends towards the proximal end.

6. The single-use safety lancet of claim 4, wherein the at least one contact wing extends towards the distal end.

7. The single-use safety lancet of claim 1, wherein the at least one contact wing comprises an arcuate contact surface contoured to receive a user's fingers.

8. The single-use safety lancet of claim 1, wherein the at least one contact wing extends obliquely from the housing.

9. The single-use safety lancet of claim 1, wherein the at least one contact wing defines one or more apertures formed therein.

10. A single-use lancet for piercing a subject's skin comprising:
an body having a lengthwise axis and housing a pressure-activated lancing mechanism for propelling a lancet along a lancing stroke parallel to the lengthwise axis of the body; and
at least one gripping surface extending outwardly from the body and operable to provide a surface for applying pressure in a direction parallel to the lengthwise axis of the body.

11. The single-use lancet of claim 10, further comprising a plurality of gripping surfaces.

12. The single-use lancet of claim 11, wherein the gripping surfaces extend at oblique angles from the body.

13. The single-use lancet of claim 12, further comprising a distal end for engaging a subject's skin and a proximal end.

14. The single-use lancet of claim 13, wherein the gripping surfaces extend towards the distal end.

15. The single-use lancet of claim 14, wherein the gripping surfaces are curved towards the proximal end.

16. The single-use lancet of claim 10, wherein the at least one gripping surface includes one or more apertures.

17. The single-use lancet of claim 10, further comprising a pair of opposed gripping surfaces.

18. The single-use lancet of claim 10, further comprising a protective cap to cover the lancing apparatus.

19. The single-use lancet of claim 18, wherein the protective cap is removably secured to the body.

20. A method for piercing the skin of a subject using a single-use lancing device comprising an elongated body housing a spring-driven lancing apparatus and at least one gripping wing extending outward from the elongated body, the method comprising:
positioning the lancing device with respect to a lancing site on the skin of the subject;
applying pressure against the at least one gripping wing; and
activating the spring-driven lancing apparatus to release a lancet along a lancing stroke.
